(19) United States
(12) Patent Application Publication
(10) Pub. No.: US 2010/0228191 A1
(43) Pub. Date: Sep. 9, 2010

(54) LOCKABLE SUPPORT ASSEMBLY AND METHOD

(75) Inventors: Jeffery B. Alvarez, San Mateo, CA (US); Enrique Romo, Dublin, CA (US); Neal A. Tanner, Mountain View, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 12/398,763

(22) Filed: Mar. 5, 2009

(51) Int. Cl. A61M 37/00; A61M 25/00

(52) U.S. Cl. 604/95.01; 604/528; 604/158

(57) ABSTRACT

Assemblies and methods related to controllably lockable support structures are described. An assembly may comprise an interface defined by two adjacent tubular structures whereby the adjacent structures may be spatially locked and unlocked relative to each other with application of a load. The tubular structures may comprise one or more spring members configured to deflect with application of a load greater than a preconfigured threshold, thereby causing a locking state of the interface to change from a first locking state to a second locking state. Embodiments are described wherein such load may be a tensile and/or compressive load. Various embodiments are described wherein an interface may be locked without application of a load and unlocked upon application of the requisite load, or locked only after application of a load.

### FIELD OF THE INVENTION

The invention relates to support structures for elongate instruments, such as catheters, and particularly to controllably and independently lockable and unlockable coupling interfaces which may comprise or be integrated into elongate instruments.

### BACKGROUND

Elongate medical instruments, such as catheters, are utilized in many types of medical interventions. Many such instruments are utilized in what have become known as “minimally invasive” diagnostic and interventional procedures, wherein small percutaneous incisions or natural orafices or utilized as entry points for instruments generally having minimized cross sectional profiles, to mitigate tissue trauma and enable access to and through small tissue structures. One of the challenges associated with minimizing the geometric constraints is retaining functionality and controllability. For example, some minimally invasive instruments designed to access the cavities of the heart have steerable distal portions or steerable distal tips, but may be relatively challenging to navigate through tortuous vascular pathways with varied tissue structure terrain due to their inherent compliance. Even smaller instruments, such as guidewires or distal protection devices for carotid intervention, may be difficult to position due to their relatively minimal navigation degrees of freedom from a proximal location, and the tortuous pathways through which operators attempt to navigate them. To provide additional navigation and operational functionality options for minimally invasive interventions, it would be useful to have an elongate structure capable of not only navigating small pathways through and around tissue structures, but also selectively locking into and maintaining a desired shape for a period of time until a desired unlocking may be executed. For example, an elongated device that is capable of being placed in an unlocked state (e.g., having a flexible elongated body) for introduction through a curved lumen into a patient's body, and also capable of being placed in a locked state (e.g., wherein it maintains the shape of the elongated body) to provide stability to the distal end when the distal end of the device has reached the treatment location to deliver therapeutic intervention, can be beneficial for various minimally invasive surgical procedures. What is described herein is a controllably lockable support assembly which may be utilized with or integrated with various types of elongate instruments.

### SUMMARY

One embodiment is directed to a support assembly for an elongate instrument, the assembly comprising a proximal tubular structure having a distal interface and a longitudinal axis as well as a distal tubular structure having a proximal interface and a longitudinal axis. The tubular structures may be sequentially and lockably coupled to each other through engagement of the interfaces. At least one of the tubular structures may comprise a deflectable spring member biased to maintain a first positional locking status of the tubular structures relative to each other, wherein upon application of a deflecting load to the spring member, a second positional locking status is achieved. In one embodiment, the distal and proximal interfaces may comprise at least one male-female type pivotal engagement, which may comprise a male aspect and a female aspect having a spring member engageable with the male aspect. The female aspect may comprise a transverse member and/or a shoulder member, and either of these members may comprise the spring member. In one embodiment, each interface may comprise two diametrically opposed male-female type pivotal engagements. The first status may be an unlocked status and the second a locked status, wherein relative motion between the two tubular structures is prevented by the engagement. In another embodiment, the first status may be a locked status and the second an unlocked status. Loading for unlocking or locking an interface may be compressive and/or tensile, depending upon the particular configuration. Loads may be applied using an actuating assembly coupled to the proximal end of one of the tubular structures, which may comprise structures such as a spindle, handle, or motor. Load applying members coupled between an actuation assembly and a tubular structure may comprise structures such as a pullwire, pushwire, or driveshaft. Load-isolating conduit may be utilized to assist in the discrete loading of one or more spring members.

In one embodiment, a third tubular structure may be added to the series, and lockably coupled to one of the other two tubular structures. Each of the interfaces between the tubular structures may be independently controllably lockable, and sequentially located interfaces may be rotationally displaced from each other by about 90 degrees.

Another embodiment is directed to a method for positioning one or more elongate medical instruments comprising inserting into a patient a first elongate instrument comprising a series of independently lockably coupled tubular structures defining a working lumen through the series, wherein an interface defined between each of the independently lockably coupled tubular structures has a locked and an unlocked locking state, and wherein switching between these states may be controlled remotely by an operator; changing at least one of the interfaces from an unlocked state to a locked state; and inserting a second elongate instrument through the working lumen of the series of independently lockably coupled tubular structures to expose a distal end of the second elongate instrument to a desired anatomical location within the patient. The method may further comprise changing at least one of the interfaces from a locked state back to an unlocked state subsequent to inserting the second elongate instrument. Each of the interfaces may be inserted in an unlocked locking state, and changing at least one of the interfaces from an unlocked state to a locked state may comprise actuating an elongate load applying member from a proximal position outside of the patient. This actuating may comprise, for example, operating an electromechanical actuator or manually operating a mechanical actuator fitting.

Another embodiment is directed to a method of minimally invasive treatment delivery, comprising inserting an elongate body into a patient body while the elongate body is in an unlocked state, advancing the elongate body such that a first portion of the elongate body assumes a first curvature, while a second portion of the elongate body assumes a second curvature; placing the first portion and second portion in a locked state, such that first portion maintains the first curvature, and the second portion maintains the second curvature; and delivering a medical instrument through a distal portion of the elongate body. Such method may further comprise placing the first portion and second portion in the unlocked state, advancing the elongate body further into the patient's body, placing at least the first portion in the locked state, and delivering a second medical treatment through the distal portion of the elongate body. Alternatively, such method may further comprise advancing a second elongate body within a lumen defined by the first elongate body, while the second elongate body is in an unlocked state; placing at least a portion of the second elongate body in a locked state; and delivering a medical treatment with the distal portion of the second elongate body.

### BRIEF DESCRIPTION OF THE DRAWINGS

FIG. 1A illustrates a diagrammatic side view of two lockably engaged tubular structures.

FIG. 1B illustrates a close up partial view of a portion of the structures depicted in FIG. 1A.

FIGS. 1C and 1D illustrate diagrammatic side views of two lockably engaged tubular structures rotating relative to each other.

FIG. 1E illustrates a view of the assembly shown in FIG. 1A, with the exception that the two tubular structures have been compressed toward each other to form a locking engagement.

FIG. 1F illustrates a close up partial view of a portion of the structures depicted in FIG. 1E.

FIG. 1G illustrates a three-dimensional side view of two lockably engaged tubular structures.

FIGS. 1H and 1I illustrate three-dimensional side views of an assembly comprising a series of lockably engaged tubular structures.

FIG. 2A illustrates a diagrammatic side view of two lockably engaged tubular structures.

FIG. 2B illustrates a close up partial view of a portion of the structures depicted in FIG. 2A.

FIG. 2C illustrates a view of the assembly shown in FIG. 2A, with the exception that the two tubular structures have been tensioned away from each other to form a locking engagement.

FIG. 2D illustrates a close up partial view of a portion of the structures depicted in FIG. 2C.

FIG. 3 illustrates a diagrammatic side view of two lockably engaged tubular structures.

FIG. 4A illustrates a diagrammatic side view of two lockably engaged tubular structures.

FIG. 4B illustrates a close up partial view of a portion of the structures depicted in FIG. 4A.

FIG. 4C illustrates a diagrammatic side view of two lockably engaged tubular structures.

FIG. 4D illustrates a close up partial view of a portion of the structures depicted in FIG. 4C.

FIG. 5A illustrates a diagrammatic side view of two lockably engaged tubular structures.

FIG. 5B illustrates a close up partial view of a portion of the structures depicted in FIG. 5A.

FIG. 5C illustrates a diagrammatic side view of two lockably engaged tubular structures.

FIG. 5D illustrates a close up partial view of a portion of the structures depicted in FIG. 5C.

FIGS. 6A-6C illustrate embodiments of a medical instrument assembly featuring a series of lockably engaged tubular structures.

FIGS. 7A-7C illustrate embodiments of a medical instrument assembly featuring a series of lockably engaged tubular structures.

FIGS. 8A-8C illustrate embodiments of a medical instrument assembly featuring a series of lockably engaged tubular structures.

FIGS. 9A-9B illustrate embodiments of a medical instrument assembly featuring a series of lockably engaged tubular structures.

FIG. 10 illustrates an embodiment of a medical instrument assembly featuring a series of lockably engaged tubular structures.

FIG. 11 illustrates an embodiment of a medical instrument assembly featuring a series of lockably engaged tubular structures, coaxially engaged with another such instrument.

FIG. 12 illustrates a diagrammatic side view of four lockably engaged tubular structures.

FIGS. 13A-13C illustrate diagrammatic side views of an embodiment of a flexible tubular structures.

FIG. 14 illustrates a method for employing a medical instrument assembly comprising at least one controllably lockable interface.

FIG. 15 illustrates a method for employing a medical instrument assembly comprising at least one controllably lockable interface.

### DETAILED DESCRIPTION

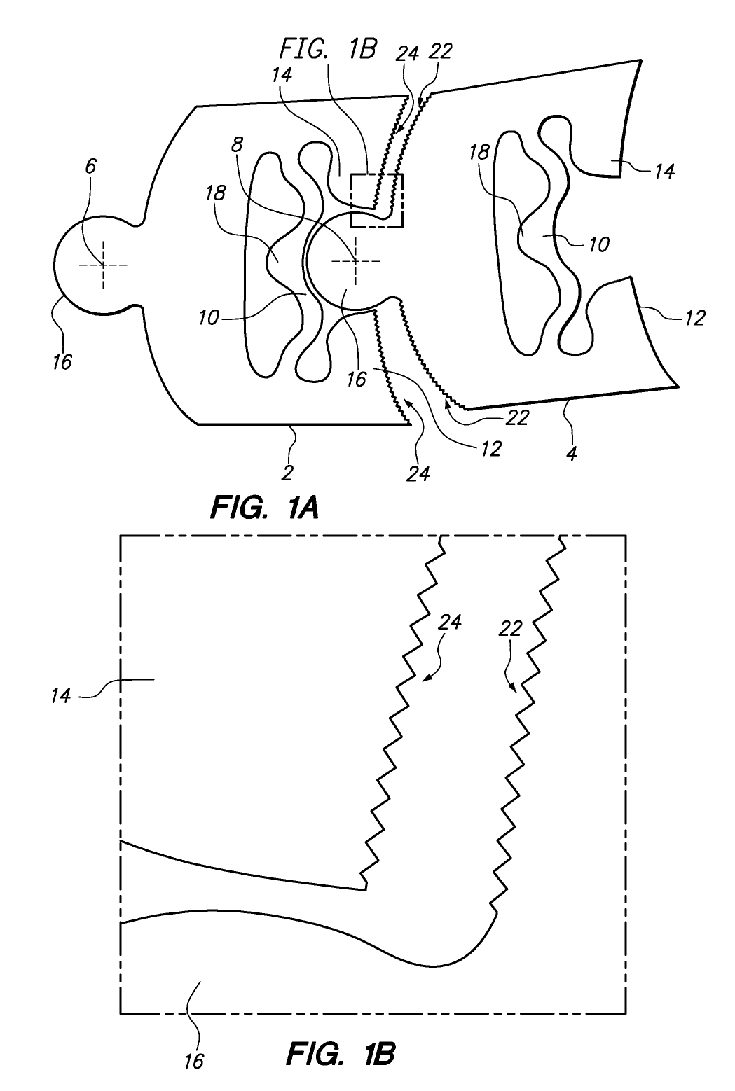

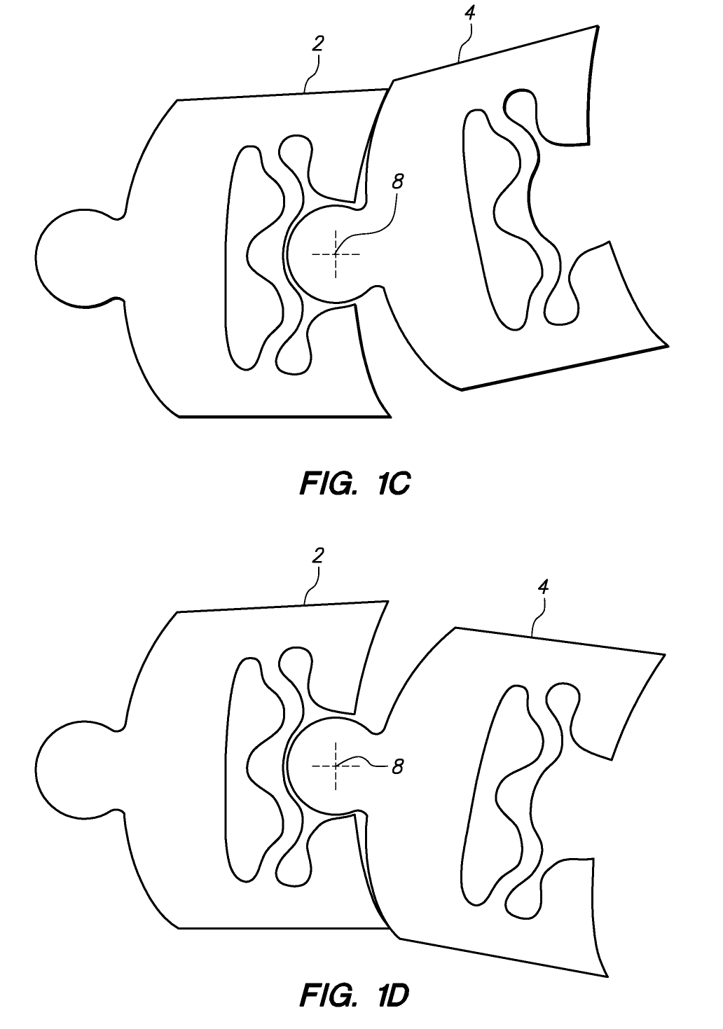

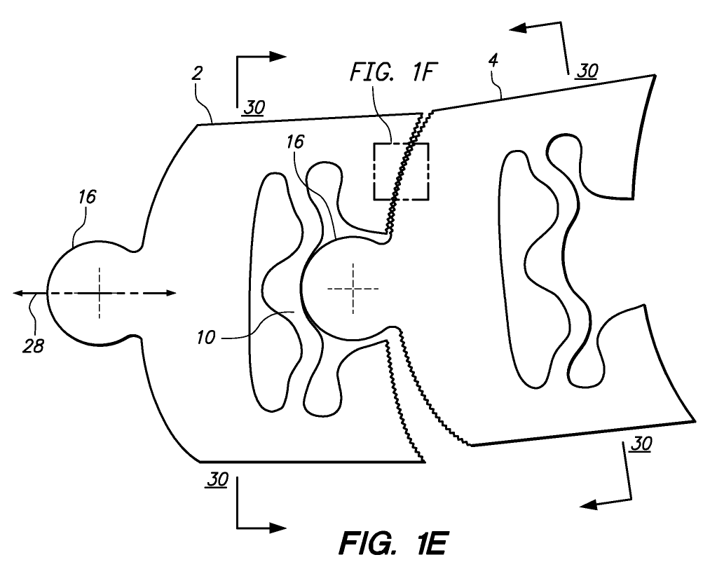

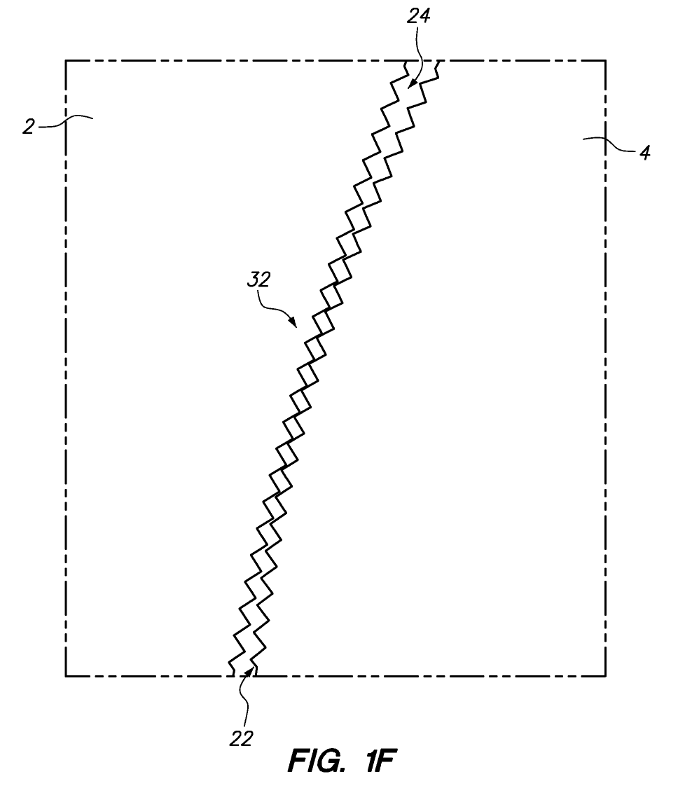

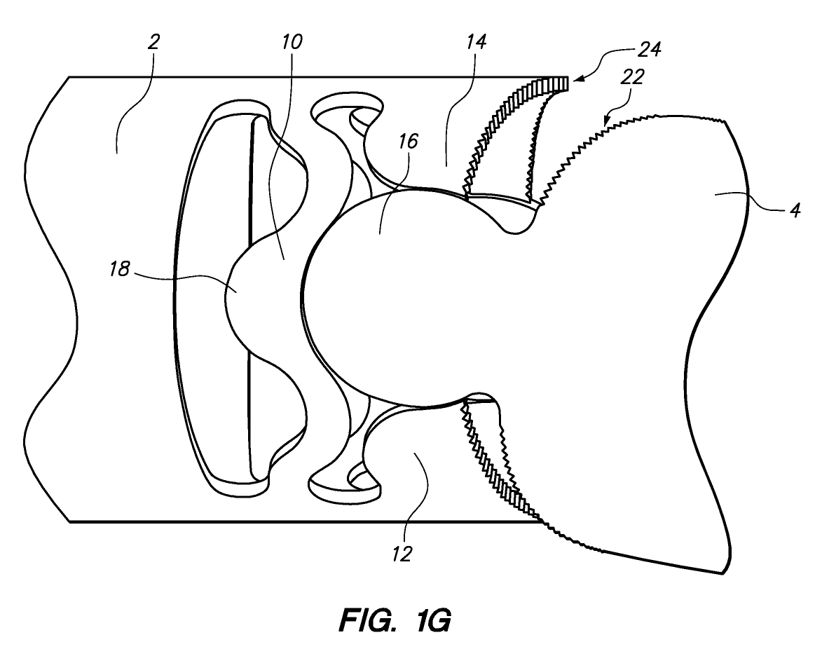

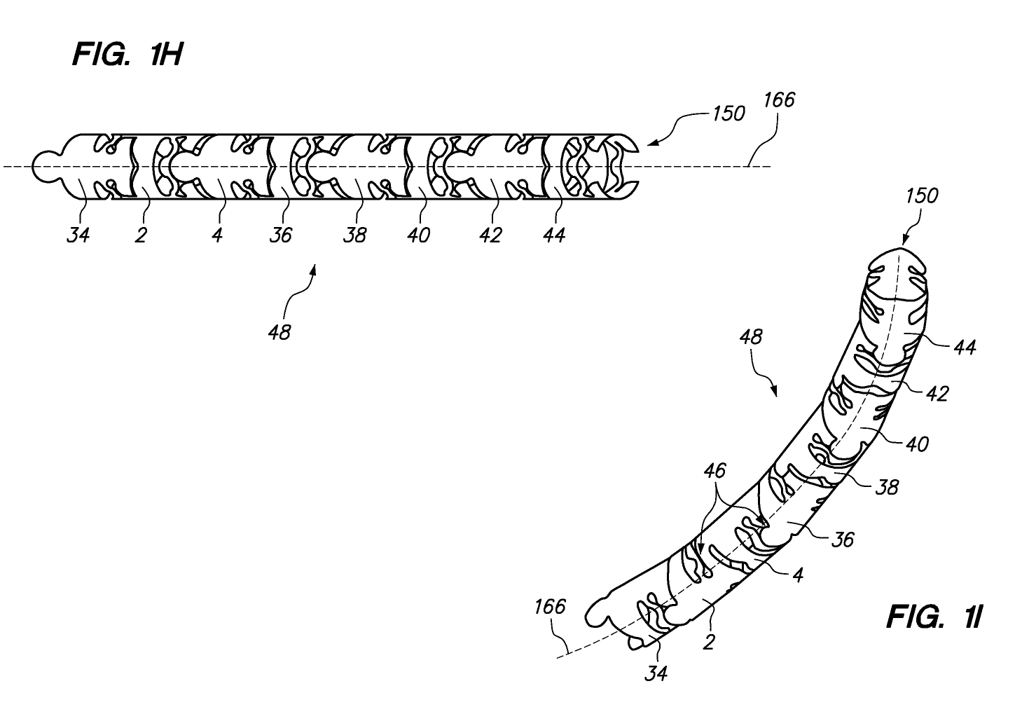

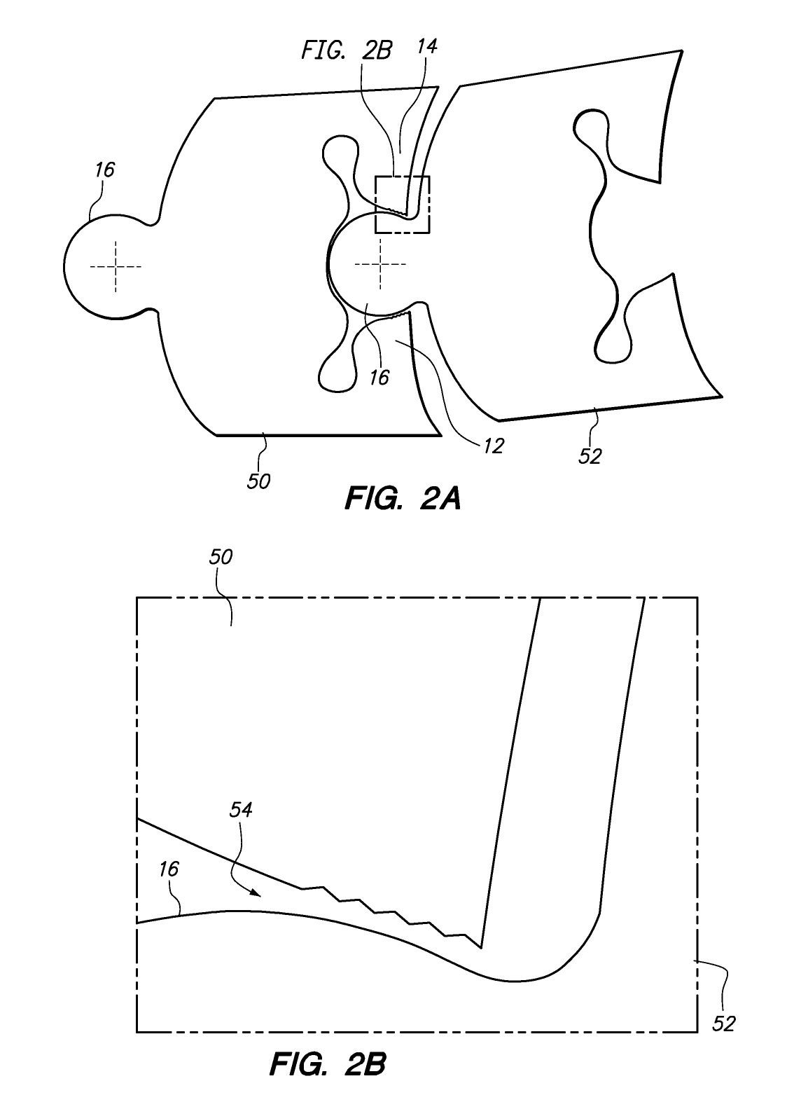

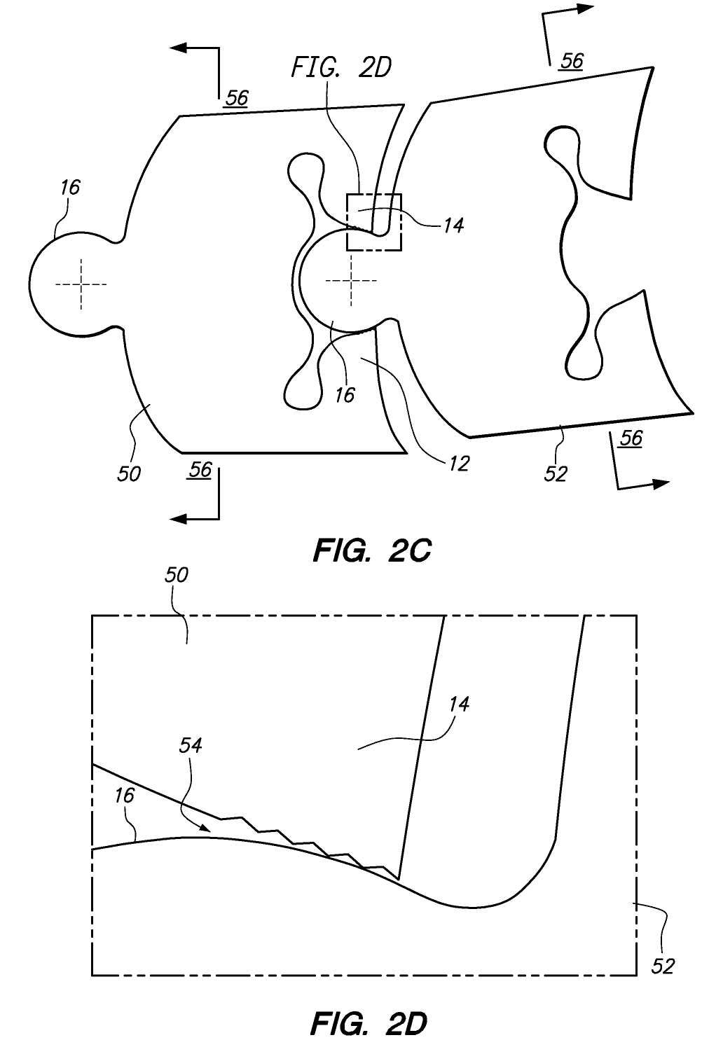

Referring to FIGS. 1A-1F, simplified diagrammatic side views of an assembly shown in greater three-dimensional detail in FIGS. 1G-1I are depicted. For simplicity and clarity of illustration and description, similar diagrammatic side views are utilized to describe embodiments associated with FIGS. 2A-5D.

As shown in FIG. 1A, two similar tubular support structures (2, 4) are coupled together. Each of the tubular support structures (2, 4) preferably is formed from a solid piece of thin-walled metal tubing, comprising a material such as nitinol or stainless steel, utilizing a process such as laser cutting or laser profiling with an automated machine such as those available from U.S. Laser Corporation of Wyckoff, N.J. In one embodiment, each of the two tubular support structures (2,4) is lasercut from the same piece of tubing. As shown in FIG. 1A, and also in FIGS. 1C and 1D, when the assembly of the first (2) and second (4) tubular support structures of this embodiment are placed in an unloaded configuration, they are free to rotate relative to each other about an axis of rotation (8) through a limited range of motion limited by physical engagement of the interfacial surfaces of the support structures (2, 4). Such freedom of motion may be desirable for embodiments of elongate instruments wherein navigation around turns or tissue structures is required. In this embodiment, each of the tubular support structures (2, 4) is configured to engage with an adjacent member in a male-female interfacial configuration wherein a substantially rounded head portion (16) comprising a tubular support structure engages a socket type space defined by two shoulder members (12, 14) and a transverse member (10).

In this embodiment, the transverse member (10) is positioned and geometrically defined to act as a spring member to bias the head (16) of the second tubular support member (4) into a position relative to the first tubular support member (2) wherein it is free to rotate, as depicted in FIGS. 1C and 1D, until a compressive load exceeding a transverse member (10) spring deflection load is applied urging the two support structures (2, 4) toward each other. A stop (18) is formed into the transverse member (10) to prevent overdeflection. Indeed, notwithstanding the non-smooth interfacial surface configurations (22, 24) created upon the tubular support structures (2, 4), the spring biasing of the transverse member (10) generally avoids contact of such surface configurations (22, 24) during rotational motion through the defined range absent application of the compressive spring deflection load. Such motion-facilitating lack of contact is depicted in the close-up view of FIG. 1B. Referring to FIG. 1E, upon application of a compressive load (30) exceeding the spring deflection load threshold, the transverse member (10) of the first depicted tubular support structure (2) is deflected toward the head element (16) of such support structure (2), allowing the head element (16) of the second depicted tubular support structure (4) to migrate farther into the first support structure (2) under the compressive load (30), thus causing engagement of the non-smooth interfaces (22, 24) of the two support structures (2, 4), and an effective “locking” spatial relationship between the two, wherein rotation is prevented by the engaged interfaces (22, 24). The engagement, or contact, (32) is shown in closer detail in FIG. 1F. The non-smooth surfaces are specifically configured to prevent relative motion upon contact (32), thus enabling a change of a locking state from “unlocked” to “locked” with contact. In one embodiment, they are laser cut to have a sawtooth type pattern, as depicted in FIGS. 1A-1G. In another embodiment, they may be otherwise treated, for example with a high-friction coating, to prevent motion with contact. In another embodiment, processing of the associated structures with tools such as lasercutters or other devices may leave deformities in the shapes that would otherwise be etched away, sanded away, or otherwise removed to a smoother finish—and in one embodiment, the mere act of omitting such smoothing process on the non-smooth surfaces (22, 24) leaves adequate frictional engagement in place at the interfaces following such processing.

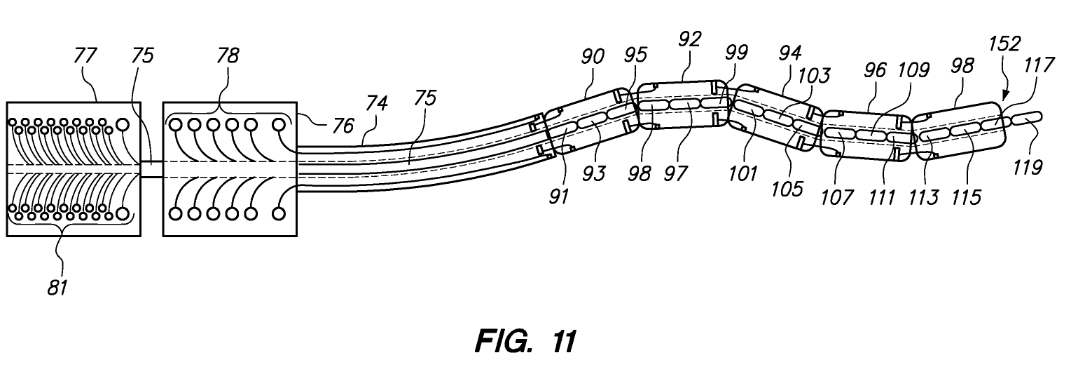

Referring to FIG. 1G, a three-dimensional detailed drawing with shadowing shows further details of an assembly of two tubular support structures (2, 4) similar to those depicted in FIGS. 1A-1F in two-dimensions. Referring to FIG. 1H and 1I, an assembly comprising a series (48) of tubular support structures (34, 2, 4, 36, 38, 40, 42, 44) is depicted, wherein adjacent tubular support structures (for example, elements 34 and 2 of FIGS. 1H and 1I) are rotationally oriented approximately 90 degrees from each other to allow for substantially omnidirectional positioning of one end of the assembly (48) relative to another end. In other words, the male-female pivotal interfacing depicted, for example, in FIGS. 1A-1F, if continued in series over multiple similar tubular support structures without rotational positioning, such as 90 degrees, between adjacent structures, would result in a range of motion something like that of a conventional bicycle chain—with a preferred plane of positioning. The rotational orientation of adjacent support structures, as depicted in FIGS. 1H and 1I, addresses this issue. For example, FIG. 11 depicts a fairly smooth “bending” positioning of the assembly (48) resulting from some rotation at each of the interfaces between the various tubular support structures (34, 2, 4, 36, 38, 40, 42, 44). Also notable in FIGS. 1H and 1I is a lumen defined by the assembly (48), which may be utilized as a working lumen construct, as discussed in further detail below. Finally, referring to FIG. 1H, a longitudinal axis (166) is defined by the assembly (48), such that the longitudinal axis of each of the tubular support structures (34, 2, 4, 36, 38, 40, 42, 44) comprising the assembly (48) is substantially aligned with the longitudinal axis (166) of the assembly (48). Notwithstanding some definitions of the word “axis”, we also use the term “longitudinal axis” in reference to the substantially arcuately-shaped axis (thus, also a longitudinal axis 166 herein) fit through an assembly (48) placed in a bent or segmentally bent configuration as depicted in FIG. 1I, wherein the longitudinal axis of each of the tubular support structures (34, 2, 4, 36, 38, 40, 42, 44) comprising the assembly (48) continues to be substantially aligned with the longitudinal axis (166) of the assembly (48). Thus FIGS. 1A-1I depict a lockable coupling configuration wherein adjacently coupled support structures are free to rotate relative to each other absent a compressive load beyond a spring deflection threshold, and with such compressive load, the adjacently coupled structures become rotationally locked relative to each other.

Referring to FIGS. 2A-2D, an embodiment is depicted wherein an interface between two tubular support structures again facilitates rotation when unloaded, but in this embodiment, only becomes locked under a tensile load greater than a spring deflection load. In other words, the embodiment of FIGS. 1A-1I is free until locked in compression, while the embodiment of FIGS. 2A-2D is free until locked in tension. As shown in FIG. 2A, as opposed to having a transverse member as the spring member in the first depicted tubular support structure (50), this embodiment uses the shoulder members (12, 14) of such support structure (50), pulled into a form of cantilevered deflection toward the second depicted tubular support structure (52) when a tensile load is applied, as depicted in FIGS. 2C and 2D. Without the requisite spring deflection load to bring the head (16) of the second support structure (52) into engagement with the shoulders (12, 14) and non-smooth surfacing created thereon (54), as depicted in FIG. 2B, the two tubular support members (50, 52) are free to rotate relative to each other. As shown in FIGS. 2C and 2D, with a tensile spring deflection load (56) applied, rotation is prevented by the contact interface between the surface of the head (16) of the second tubular support member (52) and the non-smooth surfaces (54) of the shoulder members (12, 14) of the first tubular support member (50). In one embodiment the articulating surface of the head (16) of the second tubular support member (52) also comprises texturing or non-smooth surface geometry (not shown) to promote prevention of relative motion when contact is established under a tensile spring deflection load (56) in this embodiment.

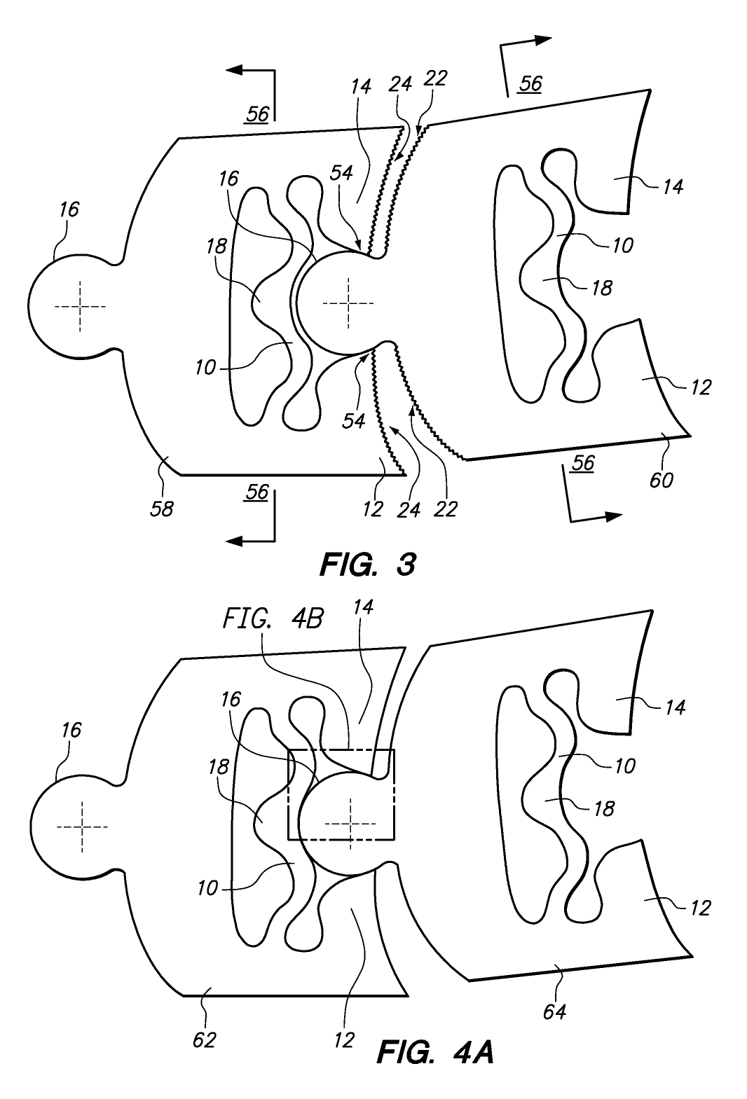

Referring to FIG. 3, an embodiment comprising aspects of both of the embodiments depicted in FIGS. 1A-1I and 2A-2D is depicted, wherein the adjacent tubular support members (58, 60) are free to rotate relative to each other unless either: a) a tensile spring deflection load (56) sufficient to cause deflection of the shoulders (12, 14) as spring members and rotating-preventing engagement as in the embodiment of FIGS. 2A-2D is applied; or b) a compressive spring deflection load (not shown) sufficient to cause deflection of the transverse member (10) as the spring member and engagement of the nonsmooth surfaces (22, 24) to prevent rotation as in the embodiment of FIGS. 1A-1I.

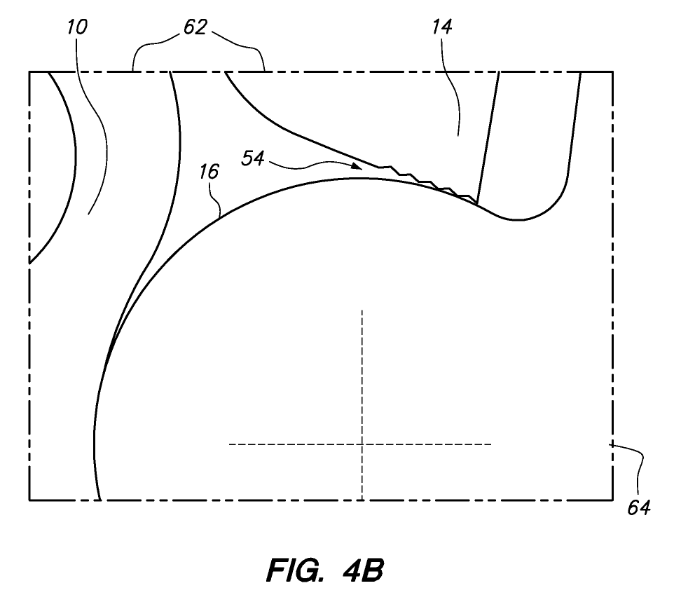

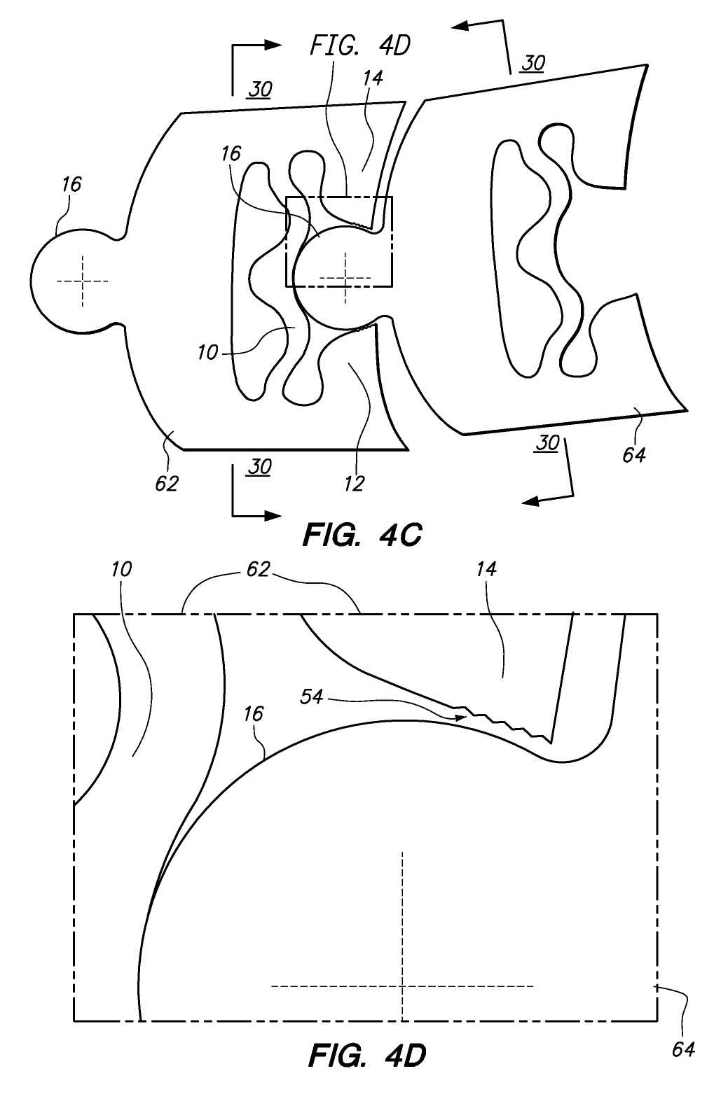

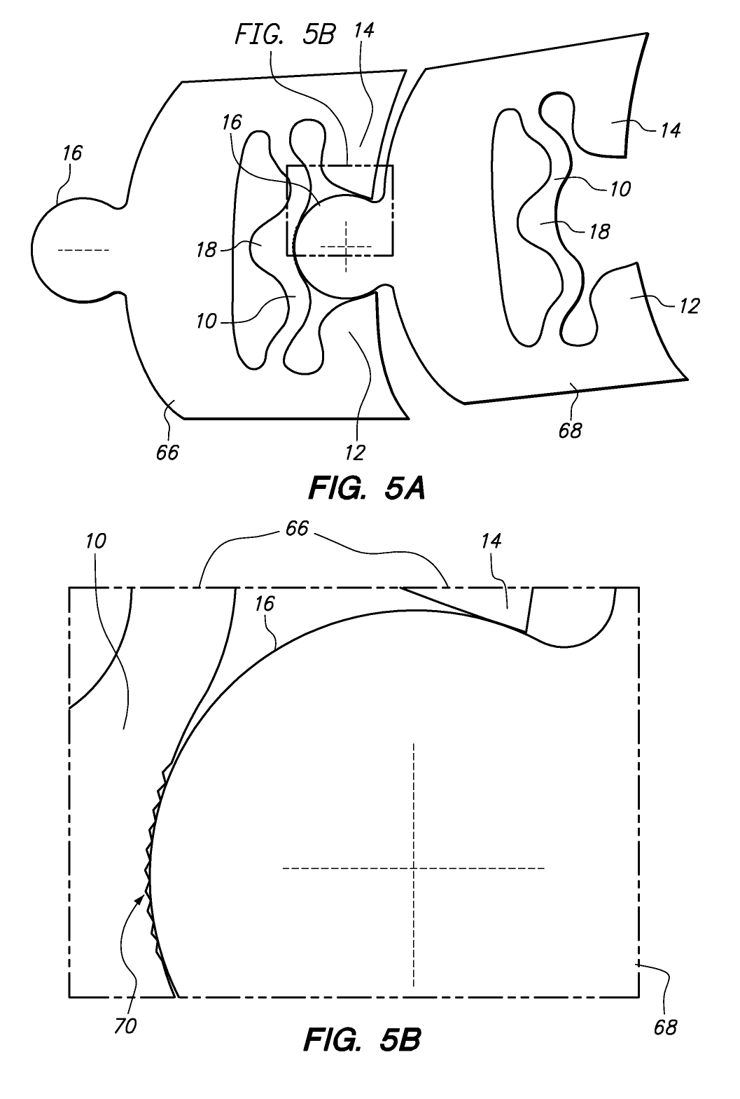

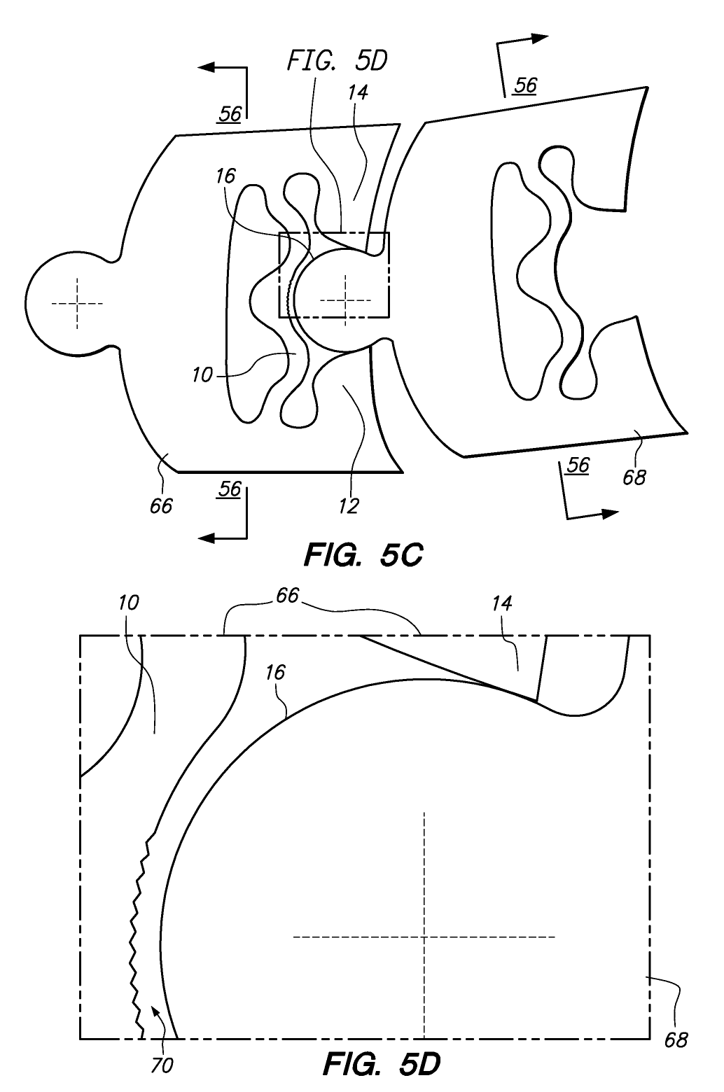

Referring to FIGS. 4A-4D, an embodiment is depicted wherein the interface is locked to prevent rotation until application of a compressive spring deflection load. Conversely, FIGS. 5A-5D depict an embodiment wherein the interface is locked to prevent rotation until application of a tensile spring deflection load.

As shown in FIG. 4A, and in close-up detail in FIG. 4B, absent the requisite compressive spring deflection load, this embodiment is configured to prevent relative rotation between the two adjacent tubular support members (62, 64) through spring biasing of the shoulders (12, 14) and transverse member (10) of the first support member (62) about the head (16) of the second support member (64) to produce engagement of non-smooth, or high-friction, surfaces, such as depicted in FIG. 4B (54). Upon application of a compressive spring deflection load (30), as depicted in FIGS. 4C and 4D, the head (16) of the second tubular support member (64) is advanced further into engagement with the first tubular support member (62) such that the high-friction interfaces (54) between the shoulders (12, 14) and the the head (16) of the second tubular support member (64) lose contact, and the tubular support members (62, 64) are free to rotate relative to each other.

Referring to FIGS. 5A-5D, a configuration similar to that described in reference to FIGS. 4A-4D is depicted, with the exception that the high-friction interface (70) is positioned at the center of the head (16) of the second tubular support structure (68), and in one embodiment, also at the adjacent surface of the transverse member of the first tubular support structure (66). As shown in FIGS. 5A-5B, absent a requisite tensile spring deflection load, relative rotation is prevented by the contact at the interface. As shown in FIGS. 5C-5D, with application of a tensile spring deflection load (56), the shoulders (12, 14) of the first tubular support member (66) are deflected and the high-friction surfaces (70, 16) are taken out of engagement, thus facilitating relative rotation of the two support members (66, 68).

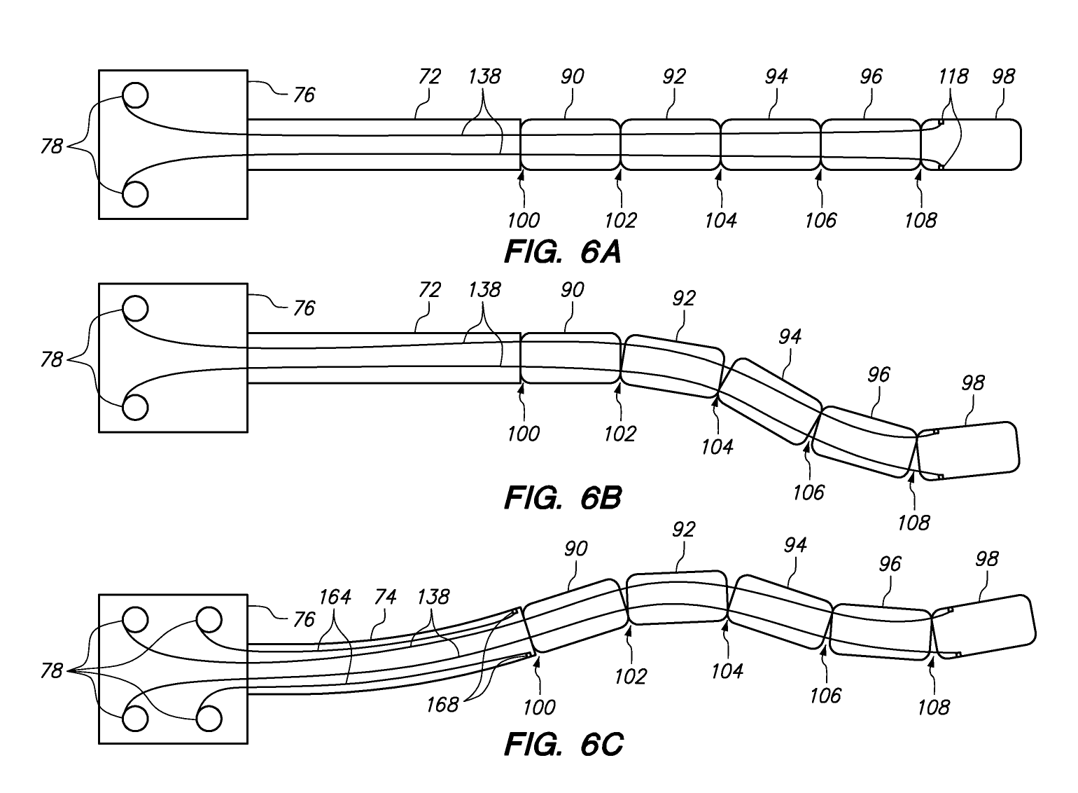

FIGS. 6A-9B depict various embodiments of implementations of five lockably coupled tubular support members integrated into an elongate medical device configuration. While these serve as illustrative example embodiments, many other variations are within the scope of this invention.

Referring to FIG. 6A, an elongate medical instrument is depicted comprising five sequentially positioned, lockably couplable, tubular support members (90, 92, 94, 96, 98). The proximal tubular support member (90) is coupled to a substantially rigid elongate tubular member (72), such as a metallic hypotube, which is proximally coupled to a proximal actuation interface (76). The proximal actuation interface structure (76) is rotatably coupled to two proximal actuation interface members (78), each of which is configured to actuate one of a pair (138) of load applying members coupled between the proximal actuation interface members (78) and a pair of termination structures (118), such as small welds or adhesive fittings, coupled to the most distal tubular support structure (98). In this illustrative embodiment, each of the four most distal lockable interfaces (102, 104, 106, 108) is configured similar to that depicted in FIGS. 1A-1F, wherein relative rotation is facilitated until a compressive spring deflection load is applied, in this embodiment by tensioning pullwires comprising the pair of load applying members (138) by rotating the two proximal actuation interface members (78), either manually by engaging a fitting or handle coupled to or comprising the proximal actuation interface members (78), or electromechanically, for example with the assistance of an electric motor coupled to the proximal actuation interface members (78). It is notable that the same load applying members that may be utilized to controllably and selectably lock certain joints may also be utilized to steer such joints through loads applied that are under the locking load thresholds for various lockable interfaces. This is true in each of the embodiments depicted in FIGS. 6A-11. For example, it may be useful form controls and electromechanical efficiency perspectives to be able both controllably steer and controllably lock various portions of an instrument assembly from a single set of proximal actuators—whether manual or electromechanical actuation is utilized. In one variation, for example, one load applying member may be gently pulled, while a diametrically opposed load applying member is gently tensioned; if the net loads at the interface are low enough, locking will not occur and the interface will thus be steerable in such fashion; should locking be desired, a cotensioning (or co-compression, depending upon whether the particular configuration is locked in compression, locked in tension, etc) of such diametrically opposed load applying members above a locking load threshold may be utilized to lock such interface in position.

FIG. 6B illustrates that freedom of rotation at the lockable interfaces may result in desired navigation and shape formation with the elongate instrument. In the embodiment depicted in FIGS. 6A and 6B, the most proximal interface (100) is rotationally fixed and not lockable. In another similar embodiment, the four most distal lockable interfaces (102, 104, 106, 108) may be configured similar to those depicted in FIGS. 2A-2D, wherein they are free to relatively rotate absent a tensile spring deflection load, and the pair of load applying members (138) may comprise pushrods, pushcables, push coils, or coil tubes to facilitate controlled proximal application of a tensile spring deflection load to lock the series of tubular support members relative to each other. In another related embodiment wherein the lockable interface configurations are configured as the interface depicted in FIG. 3, a pushrod or pushcable could also be pulled into tension to create a compressive locking as well.

Referring to FIG. 6C, an embodiment similar to that of FIGS. 6A and 6B is depicted, with the exception that a steerable tubular structure (74), such as a catheter body, is substituted for the substantially rigid elongate tubular member (72) of FIGS. 6A and 6B to illustrate that this proximal portion may also be steerable to add to the navigation complexity and capability of the instrument. Two additional proximal actuation interface members (78) are coupled to the proximal actuation interface structure (76) to facilitate actuation, manually or electromechanically, of the steering tensile members (164) which terminate distally with an additional pair of termination structures (168) to provide bidirectional steering of the steerable tubular structure (74). Other embodiments may comprise additional steering tensile members and terminations, such as a total of three or four (not shown), to facilitate omnidirectional steering of the steerable tubular structure (74).

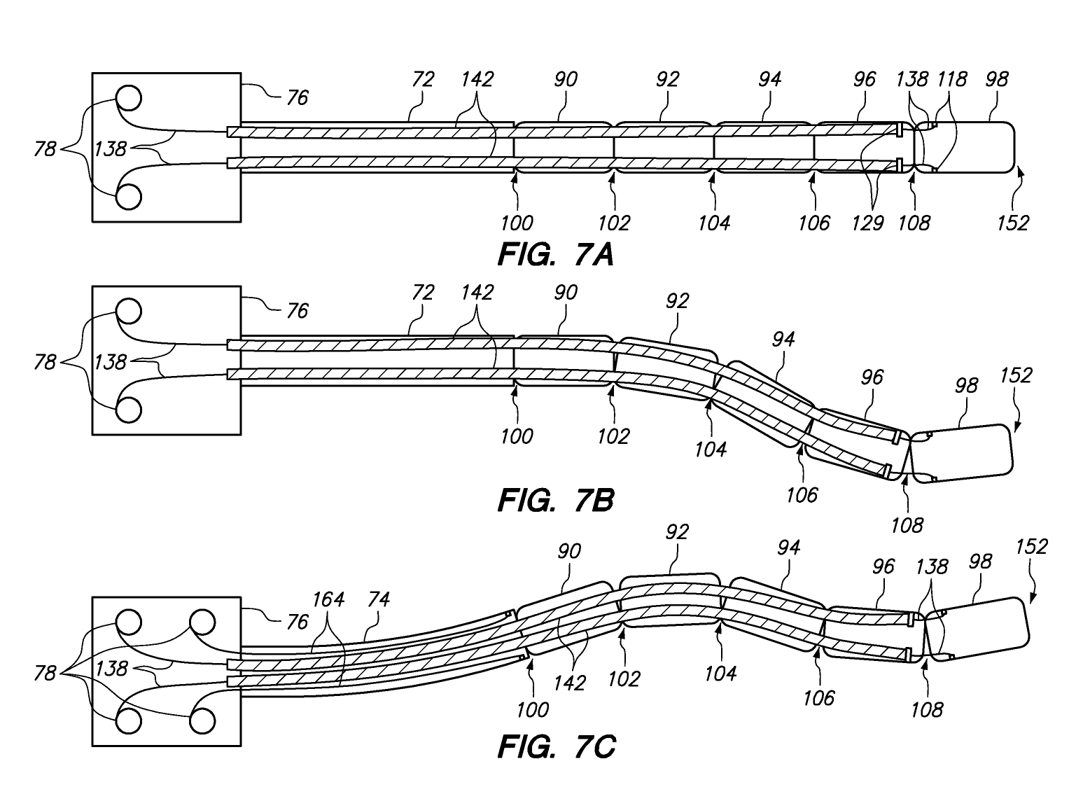

Referring to FIGS. 7A-7C, embodiments similar to those depicted, respectively, in FIGS. 6A-6C are shown, with the exception that a pair of load isolating conduits (142) is utilized to localize application of a spring deflection load for a discrete interface—here the most distal interface (108) between the distal tubular support structure (98) and the second most distal tubular support structure (96). The load isolating conduits (142) may comprise, for example, coil pipes, structural cable housings, and the like, and may be coupled between the proximal actuation interface structure (76) and a pair of load isolation termination structures (129) comprising a solder, adhesive fitting, or the like. With such embodiment, tension in the load applying members (138) only applies tension at the most distal interface (108) to lock this interface, while the remaining lockable interfaces (102, 104, 106) of the depicted embodiment remain unlocked and free to facilitate relative rotation. The embodiments of FIGS. 7B and 7C are similarly configured.

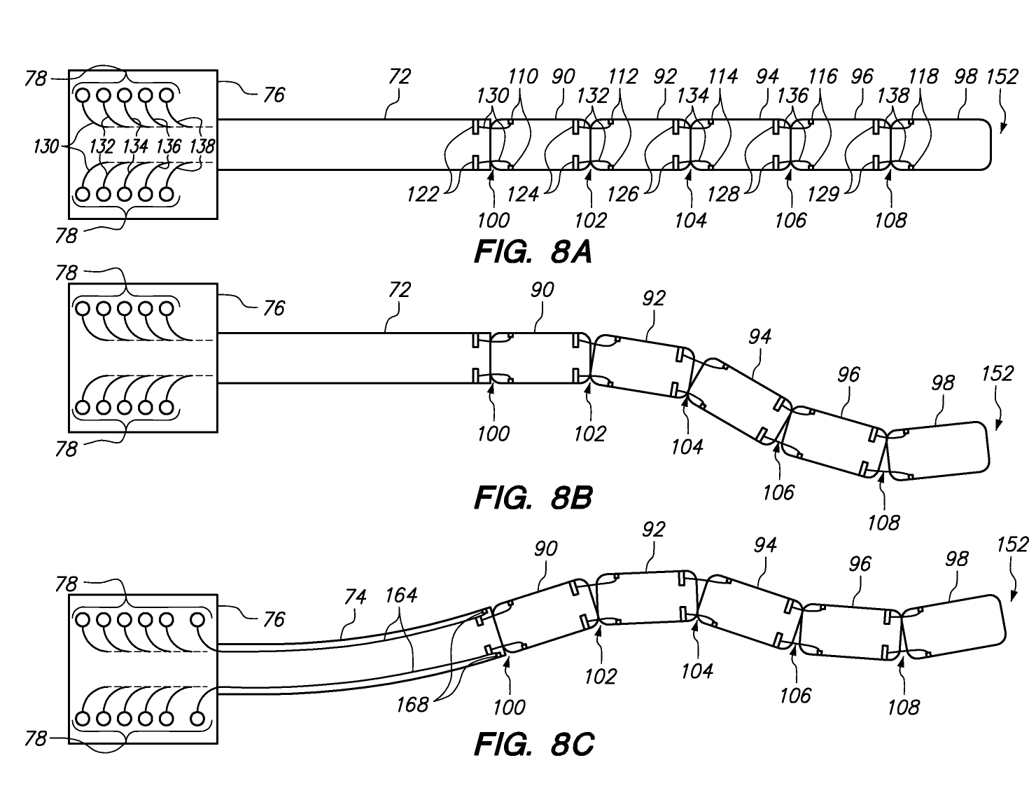

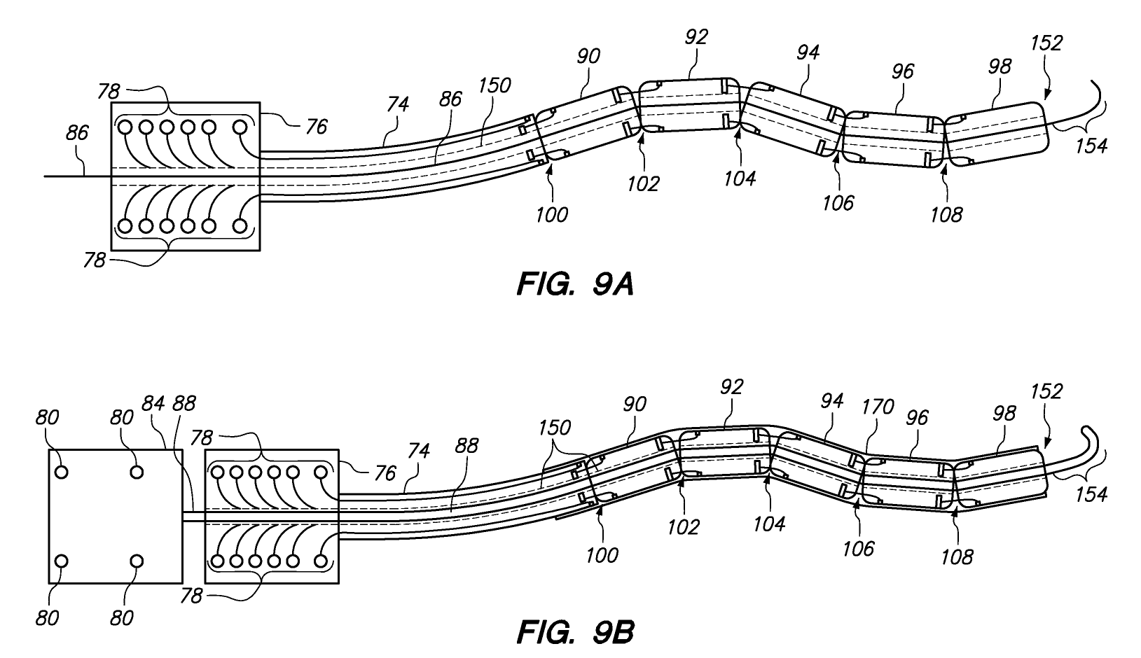

Referring to FIGS. 8A-9B, embodiments are depicted wherein, for illustrative purposes, the existence of load isolating conduits, such as those depicted in FIGS. 7A-7C (142), is denoted by pairs of load isolation termination structures (122, 124, 126, 128, 129); the hidden bodies of the load isolating conduits are intended to terminate in these embodiments at the proximal actuation interface structure (76), as in the embodiments depicted in FIGS. 7A-7C. Thus only the portions of the load applying members (130, 132, 134, 136, 138) not contained within the hidden bodies of the load isolating conduits are depicted. Thus, referring to FIG. 8A, an instrument assembly similar to that depicted in FIG. 7A is depicted, with the exception that each interface (100, 102, 104, 106, 106) is independently and discretely lockably couplable. In other words, the locking status of each, from locked to unlocked, may be independently actuated from the proximal actuation interface structure (76), such as with manual manipulation or electromechanical actuation. FIG. 8B depicts how such independent and discrete locking capability can facilitate precise turning, shape formation, and locking stability of the instrument, to, for example, lock the instrument distal tip (152) into a desired location subsequent to navigating it there with one or more rotations, or with the embodiment depicted in FIG. 8C, also including steerable tubular member (74) steering for additional navigability.

Referring to FIG. 9A, an instrument embodiment similar to that depicted in FIG. 8C is depicted, wherein a working lumen (150) defined through the instrument assembly is utilized as a controllable, repositionable, lockable conduit for a relatively small instrument, such as a guidewire (86). As shown in FIG. 9A, the distal portion (154) of the guidewire (86) may be navigated with the assistance of the lockably coupled elongate instrument assembly, from a position adjacent the proximal actuation interface structure (76). Similarly, as shown in FIG. 9B, a more complex instrument, such as a robotic catheter sold under the tradename Artisan™, available from Hansen Medical, Inc., of Mountain View, Calif., as described, for example, in U.S. patent application Ser. Nos. 11/073,363, 11/481,433, 11/637,951, and 11/690,116, each of which are incorporated by reference herein in their entirety, may be interfaced through the working lumen (150) and navigated out the distal end (152) of the lockable instrument assembly so that the distal portion (154) of the robotic catheter may be desirably navigated to nearby tissue structures for diagnostics, treatment, and the like. Also illustrated in the embodiment depicted in FIG. 9B is a sleeve layer (170) configured to at least partially encapsulate a portion of the instrument. Such sleeve layer (170) may comprise polymers, such as heat-shrink or lubricious polymers, and/or metals, such as a metal ribbon braided and/or coiled into place to form the sleeve layer (17). The sleeve layer may be configured to have many functions, such as avoiding “pinch” points between tubular or other structures, maintaining alignment of longitudinally associated members, avoiding kinking, improving friction properties relative to other nearby structures such as tissues, and/or improving imaging qualities when such structures are viewed with ultrasound, fluoroscopy, direct visualization, or other imaging modalities.

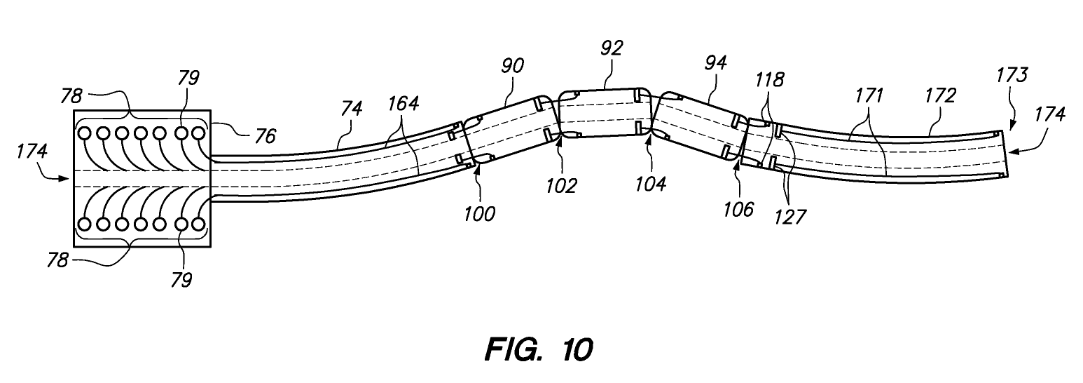

Referring to FIG. 10, an embodiment is depicted wherein two flexible elongate segments (74, 172) are interrupted by a lockable segment comprising a plurality of tubular structures (90, 92, 94) lockably coupled relative to each other. Such embodiment is configured with load isolating conduits and load applying members, similar to those described in reference to other embodiments above, such that the distal elongate member (172) may be independently steered with bending relative to its proximal end through load applying members (171) anchored distally and entering load isolating conduits (127) proximally, to enable coupling to a pair of proximal actuation interfaces (79) supported by the proximal actuation interface structure (76). The proximal actuation interface structure (76) also supports proximal interfaces (78) to facilitate proximal actuation, as described above, for positioning of each of the three tubular structures (90, 92, 94) relative to each other and relative to the two elongate members (74, 172). Further, the proximal elongate member (74) is steerable with bending relative to its proximal end through load applying members (164) that are also proximally actuatable by proximal actuation interface (78). In the depicted embodiment, a working lumen (174) remains defined through the entire assembly, to enable the use of elongate tools and other instruments, as described above, for example, in reference to FIGS. 9A and 9B.

Referring to FIG. 11, an embodiment is depicted having two lockable elongate instruments coaxially associated with each other. The outer instrument assembly depicted is the same as that depicted in FIG. 9A, with the exception that in place of the guidewire from FIG. 9A, another lockable elongate instrument has been placed through the working lumen of the outer instrument. Referring to FIG. 11, the inner instrument comprises a proximal actuation interface structure (77) similar to that (76) of the outer instrument, with the exception that the former is configured to support a larger series of proximal actuation interfaces (81) to control steerable bending of a proximal elongate member (75), as well as a series of sixteen lockably interfaced and proximally-actuated steerable tubular members (91, 93, 95, 97, 99, 101, 103, 105, 107, 109, 111, 113, 115, 117, 119). In use, the inner instrument assembly may be inserted into the outer instrument assembly while both are in an unlocked state, or while one is in an unlocked state, such that it causes the other coaxially coupled instrument to conform to its shape. Once at a desired insertion point, one or more of the segments of each instrument may be locked and/or repositioned to optimize pertinent therapy and/or diagnosis with such instrument set. The inner instrument assembly may define a small through lumen (not shown) to facilitate insertion of a small tool, such as a guidewire or needle, from a proximal location at the proximal actuation interface structure (77) to an operating theater inside of the patient at the other end of such lumen.

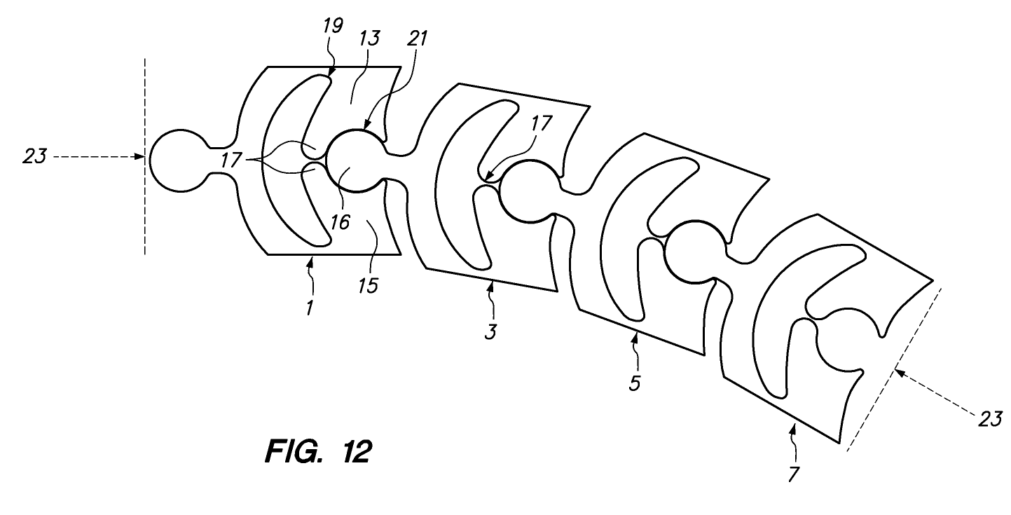

Referring to FIG. 12, a diagrammatic illustration of another controllably lockable tubular structure embodiment is depicted. In this embodiment, the shoulder members (13, 15) of each tubular member comprise distal portions (17) configured to bend the shoulder as the overall assembly is placed into compression (23), thus causing a pinching mechanical constraint at the interface (21) between the shoulder members (13, 15) and the head member (16) of the next adjacent tubular structure. Without such compressive load beyond the threshold spring deflection load, wherein the shoulder is being utilized as a spring to store energy, the head member (16) is free to rotate. In a related embodiment, the shoulder members (13, 15) may be configured to also lock the interface when the overall assembly is placed in tension enough to cause pinching at such interface (21). In one embodiment, the surfaces comprising the interface (21) may comprise nonsmooth surfaces, as described above. A series of four identical tubular structures (1, 3, 5, 7) are depicted in such a compression-lockable configuration.

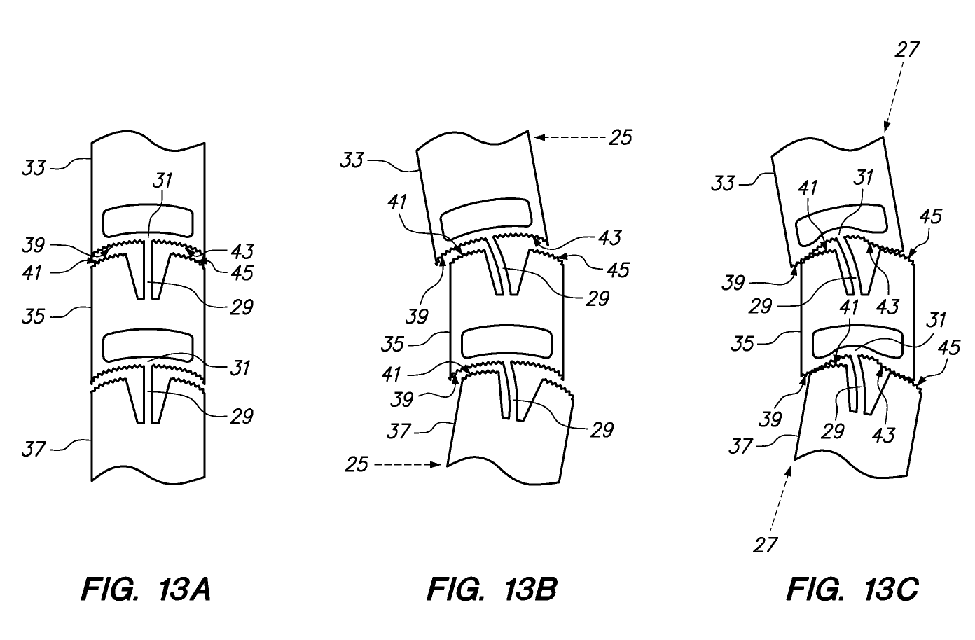

Referring to FIGS. 13A-13C, a lockably bendable tubular structure is depicted wherein three main tubular portions (33, 25, 37) are coupled by bendable connector members (29), which are coupled to the main tubular portions at bendable transverse members (31). Interfacing surfaces (39, 41, 43, 45) preferably are configured to have non-smooth surfaces for improved mechanical locking, as described above. Such a structure may be manufactured, for example, from a single piece of tubing utilizing a lasercutter. When placed under a sheer load (25), as depicted in FIG. 13B, the structure is configured to bend, through deflection most particularly at the connector members (29). When placed in compression or a combined load of compression and shear (27), the structure is configured to lock similar to some of the above-described embodiments, with the interfacing surfaces (39, 41, 43, 45) becoming mechanically engaged, thanks to the deflection of both the connector members (29) and the transverse members (31), as depicted in FIG. 13C. With such embodiment, energy is being stored in the deflected members (29, 31), and such members are acting as springs. Thus an embodiment is presented that is compressibly lockable, yet capable of unlocked bending and steering through pullwires and the like, as described above. Further, such embodiment relies upon deflection without interfacial motion between two adjacent parts, as in the aforementioned embodiments, to bend and deform as an assembly.

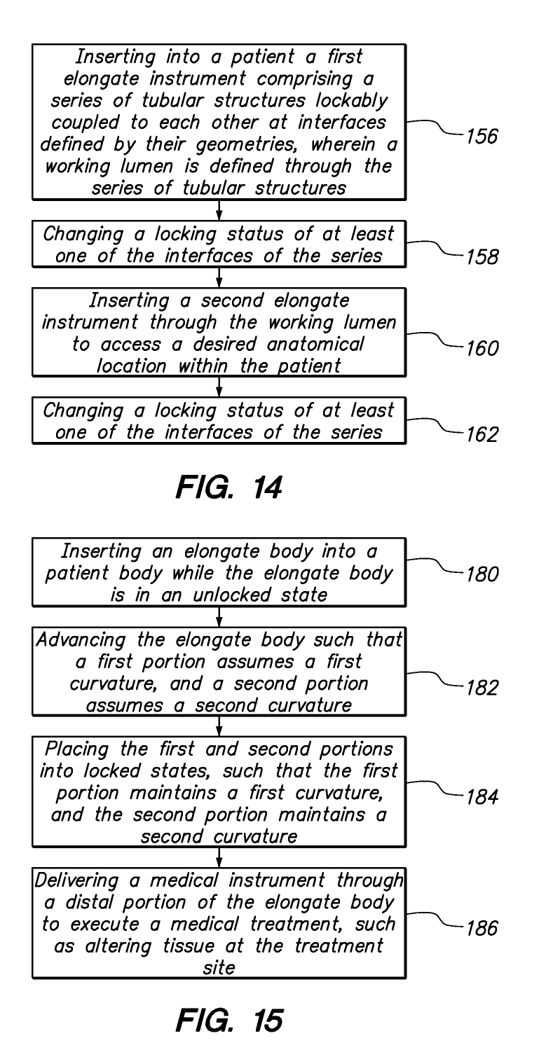

Referring to FIG. 14, a method for utilizing a selectively lockable instrument is depicted. A first elongate instrument comprising a series of tubular structures lockably coupled to each other at interfaces defined by their geometries is inserted into a patient (156). A working lumen is defined through the series of tubular structures. A locking status of at least one of the interfaces in the series is controllably changed (158). A second elongate instrument is inserted through the working lumen of the first instrument to access a desired anatomical location within the patient (160). A locking status of at least one of the interfaces comprising the series in the first instrument may be changed again (162), say from locked to unlocked.

Referring to FIG. 15, a method for utilizing a selectively lockable instrument is depicted. An elongate body is inserted into a patient body while the elongate body is in an unlocked state (180). The elongate body is advanced such that a first portion assumes a first curvature, and a second portion assumes a second curvature (182). The first and second portions are placed into locked states, such that the first portion maintains a first curvature, and the second portion maintains a second curvature (184). A medical instrument, such as a catheter, guidewire, elongate imaging device, elongate grasping device, elongate ablation device, elongate injection device, or other medical instrument, is delivered through a distal portion of the elongate body to execute a medical treatment, such as altering tissue at the treatment site (186). In another embodiment, a method may further comprise placing the first portion and second portion in the unlocked state, advancing the elongate body further into the patient's body, placing at least the first portion in the locked state, and delivering a second medical treatment through the distal portion of the elongate body. In an alternate further embodiment, a method may comprise the elements described in reference to FIG. 15, as well as advancing a second elongate body within a lumen defined by the first elongate body, while the second elongate body is in an unlocked state; placing at least a portion of the second elongate body in a locked state; and delivering a medical treatment with the distal portion of the second elongate body.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

What is claimed is:

1. A support assembly for an elongate instrument portion having a changeable longitudinal axis, the support assembly comprising:
a. a proximal tubular structure having a distal interface and a longitudinal axis;
b. a distal tubular structure having a proximal interface and a longitudinal axis, and being sequentially and lockably coupled to the proximal tubular structure through engagement of the interfaces such that the longitudinal axes of the tubular structures and elongate instrument portion are substantially aligned;
wherein at least one of the tubular structures comprises a deflectable spring member biased to maintain a first positional locking status of the proximal tubular structure relative to the distal tubular structure, and wherein upon application of a deflecting load to the spring member, a second positional locking status is achieved.

2. The assembly of claim 1, wherein the distal and proximal interfaces comprise at least one male-female pivotal engagement.

3. The assembly of claim 2, wherein the male-female pivotal engagement comprises a female aspect and a male aspect, and wherein the female aspect comprises the spring member.

4. The assembly of claim 3, wherein the female aspect comprises a transverse member and a shoulder member, and wherein the spring member comprises the transverse member.

5. The assembly of claim 1, wherein the first status is an unlocked status, such that the proximal and distal tubular structures are free to move relative to each other, and wherein the second status is a locked status, such that relative movement between the proximal and distal tubular structures is prevented.

6. The assembly of claim 1, wherein the first status is a locked status, such that relative movement between the proximal and distal tubular structures is prevented, and wherein the second status is an unlocked status, such that the proximal and distal tubular structures are free to move relative to each other.

7. The assembly of claim 1, further comprising an actuating assembly coupled to a proximal end of the proximal tubular structure, and an elongate load-applying member coupled between the actuating assembly and the distal tubular structure, wherein the load-applying member is configured to apply the deflecting load to the spring member.

8. The assembly of claim 7, further comprising a load-isolating conduit movably coupled to the load applying member and at least one of the proximal tubular member and the distal tubular member.

9. The assembly of claim 1, further comprising a third tubular structure having a proximal interface and a longitudinal axis, wherein the third tubular structure is sequentially and lockably coupled to a proximal interface of the distal tubular structure.

10. The assembly of claim 9, wherein the each of the interfaces between the tubular structures is independently controllably lockable.

11. A method for positioning one or more elongate medical instruments, comprising:
a. inserting into a patient a first elongate instrument comprising a series of independently lockably coupled tubular structures defining a working lumen through the series, wherein an interface defined between each of the independently lockably coupled tubular structures has a locked and an unlocked locking state, and wherein switching between these states may be controlled remotely by an operator;
b. changing at least one of the interfaces from an unlocked state to a locked state; and
c. inserting a second elongate instrument through the working lumen of the series of independently lockably coupled tubular structures to expose a distal end of the second elongate instrument to a desired anatomical location within the patient.

12. The method of claim 11, further comprising changing at least one of the interfaces from a locked state back to an unlocked state subsequent to inserting the second elongate instrument.

13. The method of claim 11, wherein each of the interfaces is inserted in an unlocked locking state.

14. The method of claim 11, wherein changing at least one of the interfaces from an unlocked state to a locked state comprises actuating an elongate load applying member from a proximal position outside of the patient.

15. A method of minimally invasive treatment delivery, comprising:
a. inserting an elongate body into a patient body while the elongate body is in an unlocked state;
b. advancing the elongate body such that a first portion of the elongate body assumes a first curvature, while a second portion of the elongate body assumes a second curvature;
c. placing the first portion and second portion in a locked state, such that first portion maintains the first curvature, and the second portion maintains the second curvature; and
d. delivering a medical instrument through a distal portion of the elongate body.

16. The method of claim 15, further comprising:
a. placing the first portion and second portion in the unlocked state;
b. advancing the elongate body further into the patient's body;
c. placing at least the first portion in the locked state; and
d. delivering a second medical treatment through the distal portion of the elongate body.

17. The method of claim 15, further comprising:
a. advancing a second elongate body within a lumen defined by the first elongate body, while the second elongate body is in an unlocked state;
b. placing at least a portion of the second elongate body in a locked state; and
c. delivering a medical treatment with the distal portion of the second elongate body.
